(19) United States
(12) Patent Application Publication
(10) Pub. No.: US 2005/0159812 A1
(43) Pub. Date: Jul. 21, 2005

(54) Bone-tendon-bone implant

(76) Inventors: Fred B. Dinger III, San Antonio, TX (US); Daniel R. Lee, San Antonio, TX (US); Gabriele G. Niederauer, San Antonio, TX (US); Jeffrey S. Wrana, San Antonio, TX (US)

Correspondence Address: GREENLEE WINNER AND SULLIVAN P C, 4875 PEARL EAST CIRCLE, SUITE 200, BOULDER, CO, 80301, US

(21) Appl. No.: 11/037,702

(22) Filed: Jan. 18, 2005

Related U.S. Application Data
(60) Provisional application No. 60/537,214, filed on Jan. 16, 2004.

(51) Int. Cl. A61F 2/08

(52) U.S. Cl. 623/13.14

(57) ABSTRACT

An implant for repairing soft tissue injuries is provided comprising at least one channel for receiving a soft tissue graft such as a tendon, ligament, or other soft tissue, to be implanted in a patient. The implant assembled with the graft is designed to fit into a bony defect, such as a graft tunnel, formed in bone of a patient. The implant can be biodegradable, and the implant-graft assembly has a pull-out strength sufficient to withstand everyday use during patient recovery.

### CROSS-REFERENCE TO RELATED APPLICATION

This application claims priority to U.S. Provisional Patent Application Ser. No. 60/537,214 filed Jan. 16, 2004, incorporated herein by reference to the extent not inconsistent herewith.

### BACKGROUND

In high-impact sports, ligaments are often injured through twisting of the knee or through an impact to the side of the knee. Primarily, the anterior cruciate ligament (ACL) and posterior cruciate ligament (PCL) are involved. To reconstruct these ligaments, the most common method involves a bone-patellar tendon-bone (BTB) graft, which is considered the gold standard. While the BTB graft has a 90-95% success rate, one of its disadvantages is that the tendon length may not match the original length of the ACL. Other alternatives for ACL reconstruction are the use of Achilles, hamstring or tibialis soft tissue grafts, where one or two tendon strands, autograft, allograft, or xenograft, are used to reconstruct the ligament. Defects are drilled in the knee, the hamstring graft is threaded into the bone tunnels, and interference screws or cross pins are used to fixate the graft. Market surveys show that in 2003 there were approximately 325,000 ACL/PCL procedures performed, of which 65% were BTB and 35% were soft tissue grafts.

A concern with these grafts is their long recovery times, and the fact that the fixation systems often do not encourage adequate bone growth around the grafts. Current fixation screws are fully dense and do not provide a lattice for host tissue ingrowth or loading of bioactive agents to accelerate healing. Current procedures to reconstruct the ligaments advocate an early rehabilitation protocol with immediate full range of motion, strengthening, neuromuscular coordination and early weightbearing. For example, adequate pull-out strength requirements to allow the patient to endure daily activities during rehabilitation are considered to be approximately 400-450N (Noyes, F. R., et al. (1984), “Biomechanical analysis of human ligament grafts used in knee-ligament repairs and reconstructions,” J. Bone Joint Surg. (Am) 66:344-352). Currently, the surgeon must carve or otherwise shape an autogenic, allogenic, or xenogenic bone graft so that the graft ligament, tendon, or other soft tissue can be attached. Thus an implant design is needed that can provide strong and rigid fixation of the graft and support tissue ingrowth and remodeling.

All publications and patent applications referred to herein are incorporated herein by reference to the extent not inconsistent herewith.

### SUMMARY OF THE INVENTION

This invention provides a synthetic, off-the-shelf implant designed so that a soft tissue graft can be easily attached thereto. The implant comprises at least one channel for receiving a soft tissue graft to be implanted in a patient. A “channel” can be a groove or an opening, as described herein. Soft tissue grafts are graft tendons, ligaments, or other soft tissues such as allograft, autograft, or xenograft semitendonosis or gracilis grafts, allograft tibialis grafts, autogenous or xenogenic hamstring tendons and others. A bone-tendon-bone graft (autogenous, allogenic, or xenogenic) is generally preferred for ligament reconstruction procedures due to the fact that the bone plug can be affixed in a bone tunnel. To replicate this type of construct with tendons that do not inherently possess bone blocks, such as hamstring or tibialis tendons, a bone block would need to be attached. These bone blocks can be difficult to procure, process, and manage to ensure adequate safety and quality. The implants of this invention solve these problems and allow use of tendons which do not inherently possess bone blocks.

The terms “autogenic” or “autologous” graft or “autograft” refer to a graft tissue taken from the patient's own body. The terms “allogenic” or “autologous” graft, or “allograft” refer to a graft taken from another person, such as a live donor or human cadaver. The terms “xenogenic” or “xenogenous” graft, or “xenograft” refer to a graft taken from another species, as known to the art, e.g. a pig.

The implants are preferably designed to fit into a bone defect, such as a graft tunnel, formed in bone of a patient. The implant can also be placed in a graft tunnel formed at least partially in cartilage, and in this case, it is typically affixed to bone due to the fact that the bone is mechanically stronger.

Means for securing the soft tissue graft to the implant can also be provided. Such means include component(s) selected from the group consisting of suture holes, sutures, screws, clips, rivets, pins, wires, staples, spikes, and other affixation means known to the art. In one embodiment, the implant comprises an inner portion with grooves (also referred to herein as channels) in which the graft is placed, and an outer collar which fits over the assembled inner portion and graft material to hold the graft material in place.

The implant can be porous or partially porous. Or the implant can be fully dense. The term “partially porous” includes implants that have a porous outer portion to encourage tissue growth into the implant and accelerate integrated healing of the soft tissue, and a less porous inner portion. The inner portion can be less porous (have fewer and/or smaller pores) than the outer portion, or can be fully dense, as required, to lend mechanical strength to the implant. The porous outer portion can be a porous outer layer and the less porous or fully dense inner portion can be a layer or the implant can grade continuously from an outer porous portion to an inner less porous or fully dense portion. Preferably the more porous portion constitutes about one-fourth to one-half the diameter of the implant. For example, a 10 mm-diameter implant may have about a one to about five mm, preferably about a one to about three mm, thick porous portion around the entire circumference of the device.

Porous and fully-dense materials for fostering tissue ingrowth and providing mechanical strength can be made in accordance with teachings known to the art, including those of U.S. Pat. Nos. 6,514,286; 6,511,511; 6,344,496; 6,203,573; 6,156,068; 6,001,352; 5,977,204; 5,904,658; 5,876,452; 5,863,297; 5,741,329; 5,716,413; and 5,607,474, incorporated herein by reference to the extent not inconsistent herewith.

In an embodiment of this invention, the average pore size of the more porous portion or layer of the implant can be between about 10 microns and about 2000 microns, or between about 50 microns and about 900 microns, or between about 100 microns and about 600 microns.

The implant not only holds the graft tissue in place during healing, but also provides the necessary mechanical strength to allow the patient to recover quickly and return the injury site as close as possible to its original condition.

In some embodiments, the implant comprises at least one projection over which the soft tissue graft can be looped. See, for example, FIG. 7.

The implant can also comprise means for affixing the implant to surrounding tissue. Such means include suture holes and surface features such as grooves, ridges, barbs, or threading.

This invention also includes a graft assembly comprising an implant as described above assembled for use with a soft tissue graft, in which the soft tissue graft is wrapped around the implant, threaded through a channel or channels thereof, or placed in grooves therein, and optionally secured to the implant as described above.

This invention also provides a method for repairing an injury to a soft tissue selected from the group consisting of tendons, ligaments and other structural tissues, said method comprising providing a soft tissue graft; providing an implant as described above; assembling the soft tissue graft and the implant to form a graft assembly; inserting the graft assembly into a defect in a bone; and optionally affixing the graft assembly in the defect using an interference screw, tack, rivet cross-pin, suture, or using an implant having surface features such as ridges, barbs, or threading that hold the implant in place. The implant can also be pressfit (also referred to herein as “interference fit”) into place when the implant is somewhat larger than the bone tunnel and compresses slightly when it is placed into the defect.

For example, one end of a soft tissue graft is attached to the implant, and the other end of the soft tissue graft is attached to a second implant, and each implant is inserted into a graft tunnel defect in a bone; and optionally secured within the defect.

In the case of a replacement of an anterior cruciate ligament in a patient, an ACL injury can be repaired using the methods of this invention by replacing the patient's ACL with a graft ligament or tendon or other soft tissue. The method comprises providing a graft replacement for the anterior cruciate ligament having two ends; providing two implants of this invention as described above; attaching one end of the ligament to one of the implants; attaching the other end of the ligament to the other of the implants; creating a defect in the femur to receive one of the implants; creating another defect in the tibia to receive the other of the implants; inserting one of the implants into the defect in the femur; and inserting the other of the implants into the defect in the tibia. Typically, the tibial implant is somewhat larger than the femoral implant to fit into a larger tibial bone tunnel. The implant should be secured into the defect, either by means of surface features on the implant or by means of other means as described herein for anchoring the implant to the surrounding bone.

### BRIEF DESCRIPTION OF THE FIGURES

FIG. 1 is a perspective view of an implant having a central channel through which the soft tissue graft can be passed.

FIG. 2 is a cross-section of FIG. 1 showing the outer portion of the implant material being more porous and the inner portion of the implant material being less porous.

FIG. 3 is a perspective view of an implant having a chamfered leading end to allow ease of insertion into the graft tunnel.

FIG. 4 is a perspective view of a two-part implant.

FIG. 5 is a perspective view of a two-part implant with the parts joined by means of a flexible membrane hinge at one end.

FIG. 6 is a perspective view of an implant having a channel to receive a soft tissue graft tendon.

FIG. 7 is a perspective view of an implant having multiple channels for receiving soft tissue grafts.

FIG. 8 is a perspective view of a two-part implant comprising a tapered central portion and a collar.

FIG. 9 is a perspective view of an implant having spiraled channels for receiving soft tissue grafts.

FIG. 10 is a front view of a knee joint having a grafted anterior cruciate ligament attached at either end to implants of this invention.

### DETAILED DESCRIPTION

This invention provides an implant for use with a soft tissue graft that encourages good and rapid bone growth around the graft and provides rigid fixation by anchoring the graft firmly to ensure adequate stiffness and strength during healing. These implants provide a pull-out strength for the graft of approximately 400N or greater during the healing period. The implant design of this invention allows the graft tendon, ligament, or other soft tissue to be wrapped around or otherwise attached to it. This graft assembly (comprising the implant and the soft tissue graft) is then pressed into a graft tunnel in a patient and can be secured directly via an interference screw inserted adjacent and parallel to it, or similar fixation means such as rivets, wedges, wires, cross-pins, and sutures, or surface features such as ridges, grooves, threading or barbs. The implant may also be secured by being pressfit into place.

In one embodiment, the bone-tendon-bone implant of this invention is useful for repairing knee ligaments, and can be effectively used to meet the soft tissue repair and fixation requirements of other diarthrodial joints. It provides a smooth channel for wrapping the soft tissue, and this prevents the tendon or other graft tissue from being bisected or damaged. The implant can also include small drill holes for suturing the tissue graft to the implant so as to prevent slippage.

The implant can comprise a fiber-reinforced matrix as detailed in U.S. Pat. Nos. 6,511,511 and 6,783,712 and U.S. patent application Ser. No. 10/931,474. The fiber and matrix combination is preferably selected such that the mechanical properties of the composite scaffold are tailored to optimal performance.

The implant can also contain a ceramic component suitable for buffering as detailed in U.S. Pat. No. 5,741,329, or achieving bimodal degradation as detailed in PCT Patent Publication No. WO 00/41711, or obtaining increased mechanical properties as detailed in U.S. Pat. No. 6,344,496.

Biodegradable polymers known in the art can be used to form the implants of this invention. Some examples are alpha poly hydroxy acids (polyglycolide (PGA), poly(L-lactide), poly(D,L-lactide)), poly(ε-caprolactone), poly(trimethylene carbonate), poly(ethylene oxide) (PEO), poly(β-hydroxybutyrate) (PHB), poly-4-hydroxybutyrate (P4HB), poly(β-hydroxyvalerate)(PHVA), poly(p-dioxanone) (PDS), poly(ortho esters), tyrosine-derived polycarbonates, polypeptides and copolymers of the above. Alternatively, the implant can be made of permanent, non-biodegradable materials known to the art, such as polyetheretherketone (PEEK), acetal, titanium, stainless steel, and cross-linked silicone.

The implant can be designed, in accordance with principles well-known to the art, to fully degrade over the period required for healing of the defect into which it is placed. For example, generally an ACL graft requires a period of about six to ten weeks for initial fixation and about three to six months for complete integration. The use of biodegradable implant scaffolds to which growth factors known to the art and their analogs, such as BMP2 have been added can significantly accelerate healing.

The implant can also include a surfactant (approximately 1% by weight) to further enhance the tissue ingrowth and biocompatibility of the material. Since a majority of the biodegradable polymers are inherently hydrophobic, fluids do not easily absorb and penetrate. A surfactant is incorporated into the matrix of the material at the time of manufacture so that no post-processing is required and it has no appreciable effect on the manufacturing operation or the creation of the porous structure. See, U.S. patent application Nos. 60/542,640 and 60/632,060 and subsequent patent applications claiming priority thereto.

The implant can be used to deliver bioactive agents such as growth factors, antibiotics, hormones, steroids, anti-inflammatory agents, and anesthetics in a variety of ways. These bioactive agents may also include mimetic growth factors that are osteogenic and/or chondrogenic. The growth factors, mimetic growth factors or peptides can be incorporated into to the implant, impregnated into the implant by absorption or adsorbed onto the implant during manufacture to supply an off-the-shelf product, including an implant with a tailored sustained release profile as described in U.S. Pat. Nos. 6,013,853 and 5,876,452, incorporated herein by reference to the extent not inconsistent herewith. Or, the bioactive agents can be added to the implant just prior to surgery. The implant can also be preseeded with autogenous cells or cell-containing media before implantation. By adding cells, and growth factors, the formation of the desired tissue or organ type can be improved significantly in terms of healing time and quality of repair.

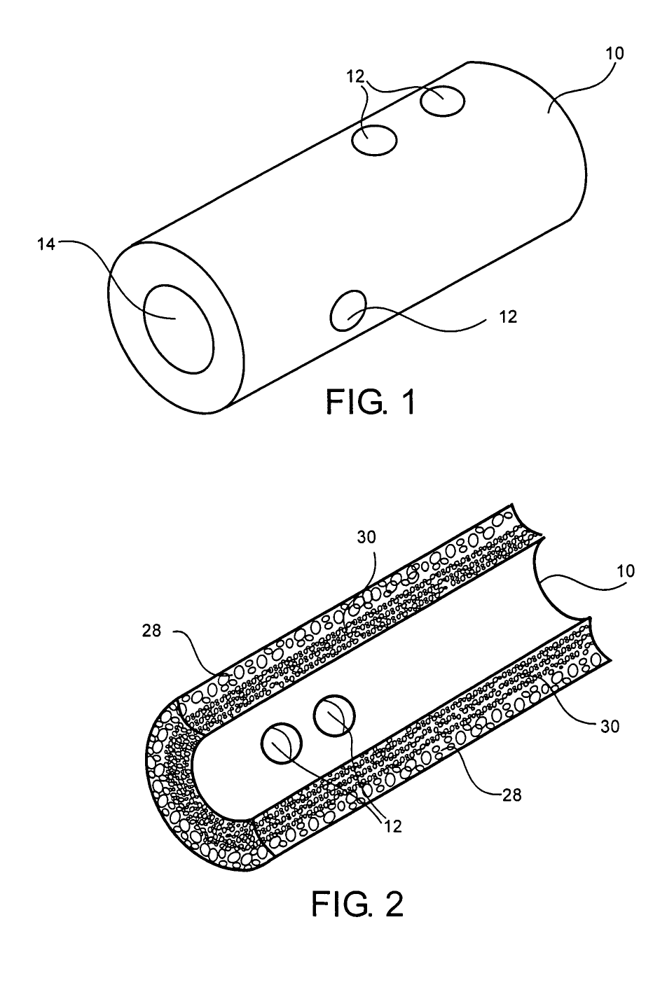

As shown in FIG. 1, one embodiment of the invention is a cylindrical implant 10 having a central channel 14 through which a graft tendon or ligament or other soft tissue can be passed. The implant also comprises suture holes 12 for tying or suturing the implant in place.

FIG. 2 is a cross-section of an implant 10 of FIG. 1 having a porous outer portion 28 of the implant material and a more porous inner portion 30 of the implant material.

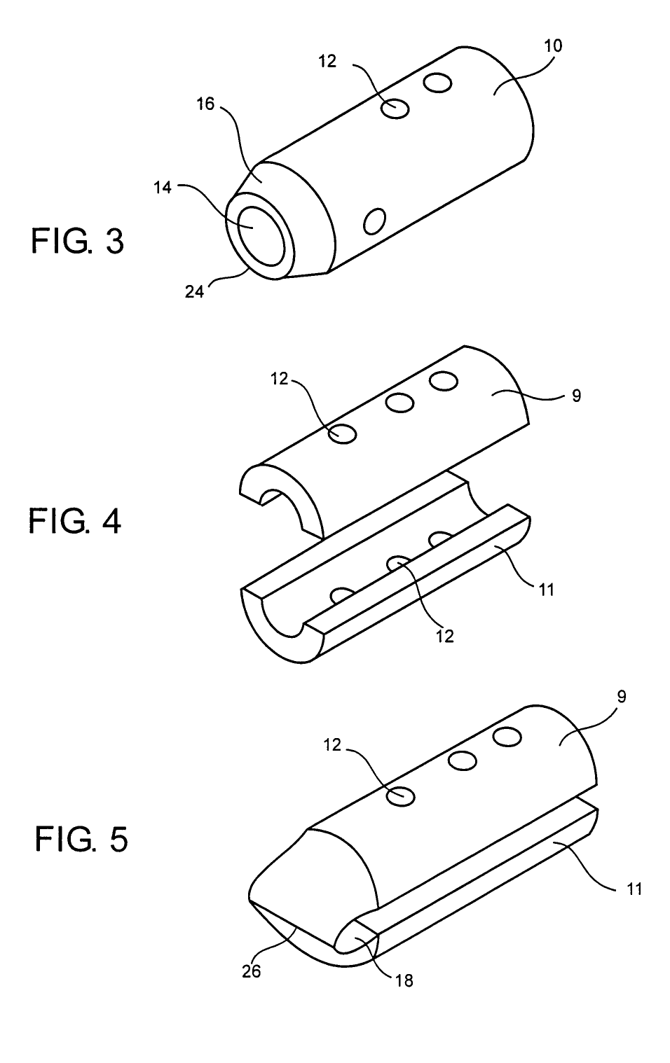

Another embodiment of the implant, shown in FIG. 3, is an implant 10 having chamfering 16 on leading end 24 to allow ease of insertion into a graft tunnel.

A further embodiment, shown in FIG. 4, is a two-part implant having a first portion 9 and a second portion 11 designed to be assembled on the surgical table or in situ. Suture holes 12 allow for the separate portions of the implant to be sutured or tied in place to tissue and/or sutured or tied to each other.

The embodiment of FIG. 5 is a two-part implant with the parts (9 and 11) joined by means of a flexible membrane hinge 26 at one end. The membrane can be a biodegradable polymer film, e.g., as described in U.S. Pat. No. 6,514,286, or other membrane known to the art bonded to the implant surface. The implant also comprises a channel 18 for receiving a graft tendon, ligament or other soft tissue.

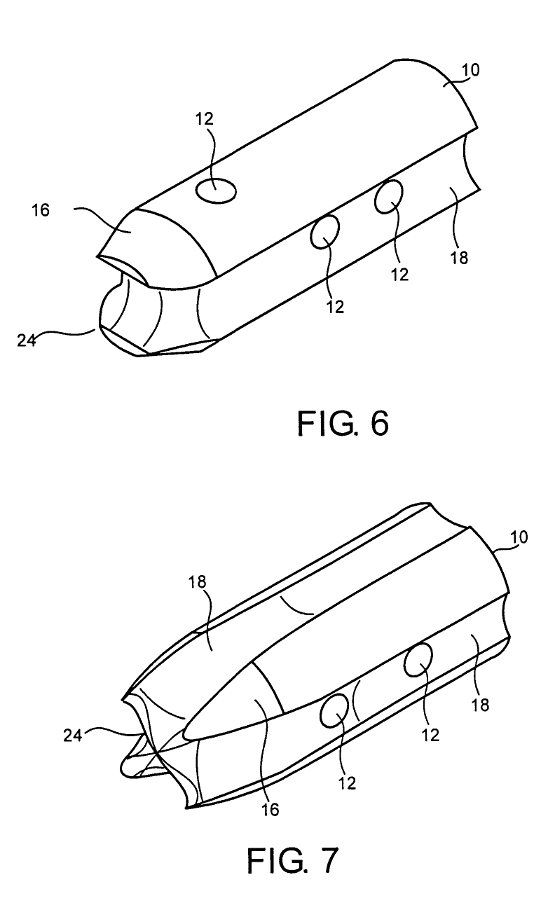

The embodiment of FIG. 6 is an implant 10 having a channel 18 completely around the leading end 24 of the implant to receive a graft tendon, ligament or other soft tissue. This allows for bone-to-implant contact on the circumferential portions of the implant 10 without interference from the graft.

An implant 10 having multiple channels 18 for receiving graft tendons, ligaments or other soft tissues is shown in FIG. 7. As an illustration, to use the implant, a semitendonosis or hamstring graft can be affixed to the implant by laying the graft in one of the channels 18 on the side of the implant and looping it over the tapered leading end 24 into the channel on the opposite side of the implant. The graft can further be secured to the implant by passing a suture through suture holes 12 and through the graft.

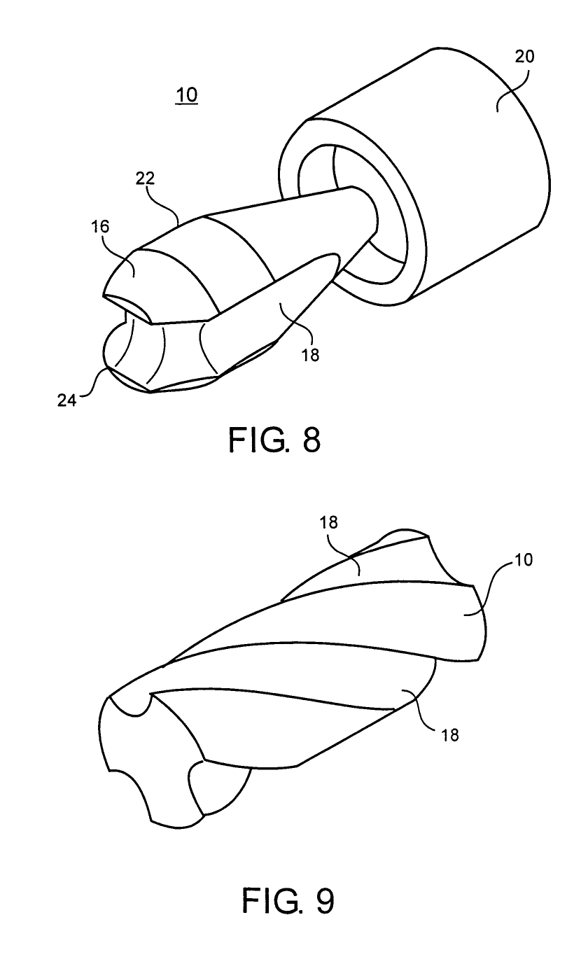

A two-part implant 10 is shown in FIG. 8. The implant comprises a tapered central portion 22 designed to fit into a collar 20. A graft tendon, ligament or other soft tissue is placed in groove 18, wrapping around the central portion 22, and is extended through collar 20. The tapering of central portion 22 allows the collar to fit snugly over the graft tendon or ligament and the central portion, thus securing the graft to the implant.

Another type of implant 10, shown in FIG. 9, has spiraled channels 18 for receiving graft tendons or ligaments.

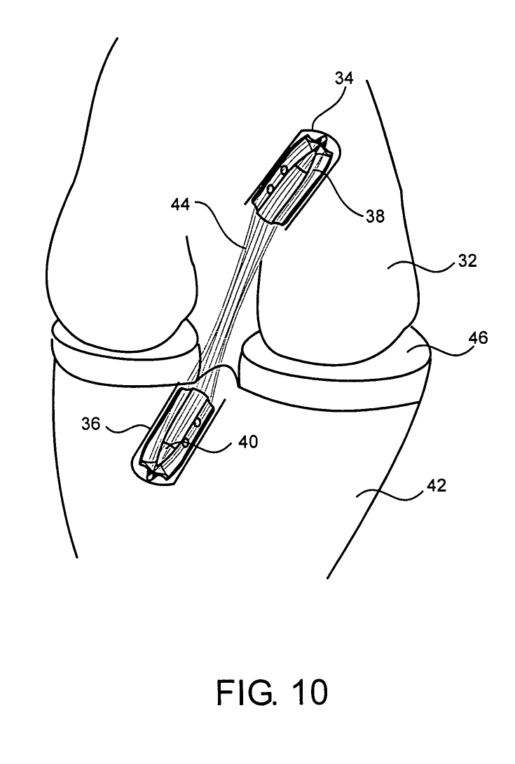

FIG. 10 is a front view of a knee joint showing the femur 32, the meniscus 46, and the tibia 42, and an anterior cruciate ligament (ACL) graft 44. A first implant 38 attached to one end of the ACL graft 44 has been inserted into a first graft tunnel 34 in femur 32, and a second implant 40 attached to the other end of the ACL graft 44 has been inserted into a second graft tunnel 36 in tibia 42.

The tendon, ligament or other soft tissue graft can be autogenic, allogenic from a live donor or cadaver, or xenogenic. Once the autogenic, allogenic, or xenogenic graft is harvested, it needs to be attached to the implant. The implant comprises at least one groove (also referred to as a channel) for placement of the graft. The implant can provide several channels that traverse the entire width or length of the implant to provide fixation. The graft can be wrapped around the implant and fixated using sutures or other mechanical attachment means such as rivets or pins. The implant is then pressed into a graft tunnel formed in the patient's bone and secured via a screw or other attachment means. The implant surface can be smooth to allow easy insertion into the bone tunnel or it can have surface features such as grooves, ridges, or barbs to increase its pullout strength. The barbs or ridges should project above the surface from about 0.2 mm to about 1 or about 2 mm. They can be shaped so that they are smooth on the side of the implant that is inserted into the defect to allow easy insertion, but provide sharp or flat obstructing features that cause increased resistance upon pullout. In the case where the implant surface is smooth, an additional means of fixating the implant into the bone tunnel may be required, such as an interference screw.

### EXAMPLE

BTB implants are assembled onto cadaver tibialis tendons on a graft preparation table by wrapping the tendons around the implants and securing them to the implants using No. 5 braided polyester suture. The implants are arthroscopically placed into cadaver knees using standard surgical technique, and secured in place with interference screws.

Knee samples are potted into testing fixtures using a fast-curing epoxy compound. Once the epoxy is cured, the samples are placed in a screw-type mechanical testing machine. The specimens are placed in tension until the graft construct fails. The implant graft assemblies are found to have pull-out strengths of 400 N and greater.

This invention has been exemplified and described in terms of specific embodiments; however, as will be appreciated by those of skill in the art, equivalent structures and methods can be used, and are within the scope of the following claims.

1. An implant comprising at least one channel for receiving a soft tissue graft to be implanted in a patient.

2. The implant of claim 1 also comprising means for securing said soft tissue graft to said device.

3. The implant of claim 2 wherein said means for securing said soft tissue graft to said device comprises a component selected from the group consisting of suture holes, sutures, screws, clips, rivets, pins, wires, spikes, and staples.

4. The implant of claim 1 wherein said soft tissue graft is selected from the group consisting of autogenic, allogenic, or xenogenic soft tissues.

5. The implant of claim 1 designed to be implanted into a bone defect.

6. The implant of claim 1 that is porous or partially porous.

7. The implant of claim 6 that has a porous outer portion and a less porous inner portion.

8. The implant of claim 1 that is fully dense.

9. The implant of claim 1 made of a biodegradable polymer.

10. The implant of claim 9 designed to fully degrade over the period required for healing of the defect into which it is placed.

11. The implant of claim 1 comprising at least one projection over which the soft tissue graft can be looped.

12. The implant of claim 1 also comprising means for affixing the implant to surrounding tissue.

13. A graft assembly comprising an implant of claim 1 and a soft tissue graft.

14. The graft assembly of claim 13 having a pull-out strength of at least about 400N.

15. A method for repairing an injury to a soft tissue selected from the group consisting of tendons and ligaments, said method comprising:
providing a soft tissue graft;
providing an implant of claim 1;
assembling said soft tissue graft and said implant to form a graft assembly; and
inserting said graft assembly into a defect in a bone.

16. The method of claim 15 also comprising affixing the graft assembly in the defect by means of an interference screw, rivet, wedge, wire, cross-pin, or suture, or surface features selected from the group consisting of ridges, threading, and barbs, or by pressfitting.

17. The method of claim 15 in which one end of said soft tissue graft is attached to said implant, said method also comprising:
providing a second implant of claim 1;
attaching the other end of said soft tissue graft to said second implant to form a second graft assembly; and
inserting said second graft assembly into a second defect in a bone.

18. The method of claim 17 also comprising affixing said first and second graft assemblies in said defects by means of an interference screw, rivet, wedge, wire, cross-pin, suture, surface features selected from the group consisting of ridges, threading, and barbs, or by pressfitting.

19. A method for replacing an anterior cruciate ligament with a soft tissue graft, said method comprising:
providing a soft tissue graft having two ends;
providing two implants of claim 1;
attaching one end of said ligament to one of said implants;
attaching the other end of said ligament to the other of said implants;
creating a defect in the femur to receive one of said implants;
creating another defect in the tibia to receive the other of said implants;
inserting one of said implants into the defect in the femur; and
inserting the other of said implants into the defect in the tibia.

20. The method of claim 19 also comprising affixing the implants in the defects by means of interference screws, rivets, wedges, wires, cross-pins, sutures, surface features selected from the group consisting of ridges, threading, and barbs, or by pressfitting.
